(19) United States
(12) Patent Application Publication
(10) Pub. No.: US 2024/0049991 A1
(43) Pub. Date: Feb. 15, 2024

(54) SYSTEMS AND METHODS FOR BED EXIT AND FALL DETECTION

(71) Applicant: Foresite Healthcare, LLC, Maryland Heights, MO (US)

(72) Inventors: George Chronis, Chesterfield, MO (US); Erik Stone, Columbia, MO (US)

(21) Appl. No.: 18/230,603

(22) Filed: Aug. 4, 2023

Related U.S. Application Data
(60) Provisional application No. 63/395,214, filed on Aug. 4, 2022.

(51) Int. Cl. A61B 5/11; A61B 5/00; G06T 7/00; G06V 40/20; G06T 7/215; G06T 7/246

(52) U.S. Cl. CPC A61B 5/1128; A61B 5/0077; A61B 5/1115; A61B 5/1117; G06T 7/0012; G06V 40/25; G06T 7/215; G06T 7/251; G06T 2207/30004; G06T 2207/10028; G06T 2207/30196

(57) ABSTRACT

Systems and methods for using depth camera imagery to identify someone intending to leave a bed or chair before the bed or chair is exited allowing intervention where necessary to prevent falls before they occur. Such systems can also be used to detect falls in other specific situations such as those which involve falls partially obscured by furniture, doorways, or other objects in the room.

### CROSS-REFERENCE TO RELATED APPLICATIONS

This application claims the benefit of U.S. Provisional Pat. App. No. 63/395,214, filed Aug. 4, 2022, the entire disclose of which is herein incorporated by reference.

### BACKGROUND OF THE INVENTION

### Field of the Invention

This disclosure is related to the field of healthcare services, including but not limited to hospital and long-term care, and particularly to systems and methods aimed at increasing the detection and inhibition of falls. The systems and methods contemplated herein primarily utilize depth camera imagery, without reliance on other sensors, to detect attempted bed exit and partially obscured falls.

### Description of the Related Art

One of the most dangerous things for patients in an acute health care setting (like a hospital), in a chronic health care setting (like a skilled nursing facility), and even at home, is the danger of falls. This is particularly true for the elderly, as falls are the leading cause of injury for adults over the age of 65, with the risk increasing with each additional year of age. By some estimates, approximately one-third of the elderly will suffer some sort of fall each year, and of those, approximately one fourth (25%) to one third (33%) will suffer moderate or severe injuries.

Even more concerning, those injuries can directly or indirectly result in death. Directly, falls result in more than 30,000 deaths annually in the United States (400,000 worldwide) in individuals over the age of 65, with “accidents,” including falls, representing the eighth leading cause of death, in large part due to the risk of traumatic brain injury which can occur during a fall. Indirectly, falls can result in decreased independence, increased pain, reduced mobility, and an overall reduction in the quality of life. These, along with the possible need for surgery and pain relief medication have each been paired with an overall reduction in life expectancy. Thus, fall prevention and inhibition in the elderly has become an important issue for care facilities of all types.

Falls are not limited to the elderly, however, and even otherwise able-bodied individuals are a higher risk of falls in healthcare settings particularly following medical procedures, accidents, heart attacks, or other non-fall events. Procedures, treatments, and medications (including pain killers) can make patients confused and dizzy and can cause bouts of semi-consciousness, nausea, or vertigo, increasing risk of falls. This risk is in addition to any increased risk due to whatever condition or event lead to the acute stay, which may already be causing muscle weakness, poor cardiovascular or pulmonary function, visual accuracy problems, pain, or other issues.

Further, even the individual's presence in a hospital, skilled nursing facility, or other healthcare environment on its own increases the risk of falls. For sanitary purposes and ease of cleaning, floors in these facilities are typically tiled—smooth hard surfaces without the cushioning of carpet—and often slippery. Rooms are often filled with equipment, including cords, tubes, and wires, adding additional dangers to even a short walk to a restroom or between a bed and chair. Perhaps most obviously—these facilities are not a “home environment” for most patients, and so any reduction of falls as a result of familiarity with the environment is lost.

In an acute healthcare setting, falls can prolong otherwise short-term stays. As a result, healthcare facilities take considerable care in assessing fall risks and in providing, where possible, systems for detecting or inhibiting falls. These include the use of relatively straight-forward systems and procedures which inhibit a patient from being able to put themselves in a position to fall. For example, bed rails on hospital beds inhibit a patient from rolling off the bed or getting up when they should not. Further, a requirement that a patient be moved by wheelchair when in the facility reduces the possibility of the patient falling by stopping the patient from standing or walking within the facility. While these can be very effective, these systems also provide direct inhibitions on a patient's autonomy when they are within the facility and often invoke negative responses.

In order to try and increase autonomy in the acute care setting, many facilities use systems that passively monitor the occurrence of falls, or that detect when the likelihood of a fall increases. In this way more inhibitive measures may be limited to more vulnerable patients and more patient freedom may be maintained. Many hospitals attempt to detect falls, or predict falls before they occur, through the use of sensors associated with a patient. In the most straightforward form, these systems utilize computer models to analyze a patient's movement to determine how stable they are analytically, and then assign increased monitoring or reduction measures to those at increased risk. More complex systems attempt to monitor and look for falls at times when they are more likely. In many cases, this occurs at bed exit. Bed exit is an activity with an increased risk of fall for virtually every person in an acute care setting. Going from a prone position to a sitting position and then to a standing position requires substantial muscle coordination and balance. Further, the prone position essentially requires no balance or strength (which is why it is so common in an acute care setting) making the transition from prone to standing a high risk activity when it comes to falls.

Bed exit systems typically work on one of two principles. Simpler systems simply seek to look for a patient no longer being in bed and to potentially have fallen. To put it simply, the systems try to detect a prone patient on the floor, and then infer that a fall has occurred. More complex systems attempt to detect that a patient is either getting up, or is falling relative to a bed, to give more advance warning of a potential fall or quicker response to an actual fall. These later systems which use attempted bed exits have the goal of preventing falls before they occur. Embodiments of such a system are contemplated in, for example, U.S. patent application Ser. No. 16/942,479 and U.S. Pat. No. 10,453,202, the entire disclosures of which are herein incorporated by reference.

While concern about falls at acute care facilities is certainly intended to improve care for patients, there are financial considerations for the facilities as well. Falls for hospitalized patients are believed to represent 30-40% of safety incidents within any hospital and will generally occur at a rate of 4-14 for every 1,000 bed days at a hospital. For even a relatively small facility, this can lead to multiple fall incidents every month, and they can be a daily occurrence for a large institution. The problem is exacerbated because falls are often seen as preventable and, therefore, falls can result in penalties to the hospital in the form of reduced governmental recognition for quality of care. They can also be a source of malpractice lawsuits.

Beginning in October 2008, Medicare stopped reimbursing hospitals for specific instances of this kind of “error.” The Centers for Medicare & Medicaid Services drew up a list of ‘reasonably preventable’ mistakes, termed ‘never-events’. After that date, falls in hospitals were no longer reimbursed by Medicare. On Jun. 1, 2011, Medicaid followed Medicare's lead in no longer reimbursing hospitals for ‘never-events’, including falls. Additionally, the Affordable Care Act imposes payment penalties on the twenty-five percent (25%) of hospitals whose rates of hospital-acquired injuries due to falls are the highest.

To counter this, use of bed exit alarms has increased because bed exit can be a particularly high source of falls. Where a patient has been deemed a fall risk, the patient can be placed in a bed, either with a bed exit alarm affixed to it, or in a bed that has a bed exit alarm which was incorporated into the bed at the time of manufacture. When a patient exits a bed where an alarm is installed, an alarm goes off allowing the potential of a fall to be rapidly identified and responded to.

The most obvious problem with most existing bed exit alarms is that they do not solve the problem of preventing or inhibiting falls after arising from a bed or chair. Instead, they merely detect that a patient has left their bed and, therefore, can detect that there is an increased risk of fall because they are now standing and/or ambulating. While a patient that has left their bed is clearly at an increased risk for a fall, they are at risk for such fall the instant they leave their bed. Thus, existing bed exit alarms effectively act to notify personnel that an individual is at a dramatically heightened fall risk only after they are at such risk.

As an individual with a high risk of fall is very likely to fall quickly after leaving their bed or chair or even as they are leaving it (before they have even had a chance to ambulate), by the point a prior alarm goes off, the fall (and resulting damage) is likely done. Thus, these systems act more to quickly detect that a fall has occurred and minimize it's impact, than to inhibit the likelihood of one occurring in the first place. While fall detection is valuable to reduce risk of long term damage from the fall due to quick response, the fundamental problem of not inhibiting the fall in the first place, is left unsolved by many prior systems and this goes a long way to explain why conventional systems only decrease falls by about twenty percent (20%) according to current statistics.

Bed exit alarm systems also have other flaws. First, they suffer from a significant rate of false negatives—alarms failing to go off when a patient has exited—which obviously defeats the purpose. They also suffer from substantial false positives—alarms going off when a patient has not left bed but is instead just moving or rolling over, or even just sitting up for a bit. These must be treated as true positives and investigated in prior systems, taxing healthcare employee resources.

Further, where a facility does not provide alarms on every bed, staff must also attempt to assess those patients with the greatest need, which should correlate with fall risk, and provide alarm system beds to those patients to most effectively reduce the incidence of falls at the facility overall. However, most fall risk assessments are highly subjective, based upon either the individual's own perceptions of their risk, or the perceptions of health care employees, which may be based upon a limited history. Even where assessments are grounded upon more objective criteria such as gait analysis, they are often based upon an individual's prior medical history and not immediate review, which may (or may not) be indicative of their risk of falling at the current time. For example, it is difficult to predict any patient's resultant balance when coming off of anesthesia versus their normal balance making any historical determination irrelevant to the present risk. In effect, the fall risk assessment in prior systems is generally determining who is more likely to need assistance and the bed alarm is simply trying to indicate when.

In general, this combination, coupled with a healthcare facility's very reasonable desire to decrease liability, causes facilities to potentially overestimate falling risk, resulting in patients often being confined to wheelchairs or beds when perhaps they do not need to be, receiving assistance from staff every time they wish to leave bed to make sure that they don't fall, and with staff being bogged down taking regular reassessments of patients to allocate limited fall risk resources. These significantly drain staffing resources. Further, as reassessments are time consuming and ordered with a regularity (often every four to eight hours) that prevents any real change from being documentable, such reassessments become a low priority for overly tasked medical staff, and are often not performed or not performed well, making them ineffective.

The alternative is to have bed exit alarms placed upon every bed. While this removes concerns of effectively triaging patients and providing alarms only to those patients at the highest risk (or perceived highest risk) of falling, it does so at great cost, and would also lead to a substantial number of false positives (alarms going off where there are no bed exits) and true positives (people exiting beds) where there are actually no substantial concerns of the patient falling. While the latter issue can be somewhat improved by manually turning certain alarms off for patients with less risk, this obviously defeats the purpose of having universal alarms installed in the first place, and effectively returns the “every bed” option to one of using alarms based upon subjective criteria.

Still further, hospital beds are already complex systems and needing to add additional sensors to them to help detect bed exits can continue to increase medical costs both directly and through increased ongoing maintenance. Bed sensors typically need to be positioned on top of the mattress (under bed linens), or under the mattress, to detect the patient accurately. As bed linens, and even the mattress itself, in a hospital setting often need to be regularly cleaned and disinfected, the inclusion of a bed sensor as a part of the mattress or linens can make these processes substantially more difficult. Thus, most bed sensors are actually inserts that go on the bed in addition to the mattress and linens. This can lead to bed sensors being misplaced or not reinstalled in the correct manner or location when the bed is turned over from one patient to another. In such a situation, the sensor may operate sub-optimally or not at all when it is still being relied on for fall safety.

Because basic privacy concerns, and manpower issues, will generally prevent institutional personnel from watching every patient all the time, automated “visual” systems have been proposed to try and both assess fall risk and to detect falls. U.S. patent application Ser. No. 13/871,816, the entire disclosure of which is herein incorporated by reference, provides for a system for fall detection and risk assessment which externally analyzes gait parameters of a patient to evaluate both their likelihood of fall risk and to notify a caregiver if a fall is detected.

The systems described in U.S. patent application Ser. No. 13/871,816 utilize a depth camera or other device which can obtain depth image data to analyze an individual's gait. Image analysis, such as is described in that application, effectively requires 3-Dimensional (3D) image data which is why a depth camera is used. Image analysis in this fashion, which is often referred to generally under the moniker of “machine vision” can be very valuable in fall risk assessment as certain elements of gait, and changes in gait, can indicate increased likelihood of falling. Further, certain actions in a gait (such as the motion of stumbling which is quite different from the motion of walking) can be immediate indicators that a fall has occurred or is occurring. Machines can generally automatically detect that such a fall has occurred based on the movement of the patient which is detected as motion akin to a fall and immediately notify caregivers to come to their aid. However, these systems typically are designed to analyze ambulation. Once a patient is ambulating, the risk for falls is clearly higher than it is before a patient is ambulating. Thus, systems such as these, while good at predicting a future fall risk and detecting the occurrence of a fall, are not good at inhibiting a forthcoming fall.

### SUMMARY OF THE INVENTION

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, what is needed are systems and methods for identifying someone intending to leave a bed or chair before the bed or chair is exited, evaluated in real time, to allow intervention where necessary to prevent falls before they occur and which don't rely on systems tied to the bed itself. These types of patient actions are referred to herein as “attempted” bed exits where the user attempts to exit the bed, but where intervention can preferably be supplied before they actually do so. Such systems can also be used to detect falls in other specific situations such as those which involve falls partially obscured by furniture, doorways, or other objects in the room.

Described herein, among other things, are systems and methods for using a depth camera for detecting a patient attempting to leave a furniture object, the method comprising: obtaining a merged point cloud from an image of said depth camera, said merged point cloud being indicative of a human on a furniture object; reviewing said image to locate a within said merged point cloud a skeleton point cloud indicative of said human; defining an edge of said merged point cloud which is not part of said skeleton point cloud, said edge being generally linear; monitoring said merged point cloud for said skeleton point cloud to move and, by moving, break said edge; and using said break to determine that said human is attempting to leave said furniture object.

In an embodiment of the method, the furniture object comprises a chair.

In an embodiment of the method, the furniture object comprises a bed.

In an embodiment of the method, during said using, said determination involves deciding if a particular portion of said skeleton point cloud moved and broke said edge.

In an embodiment of the method, the particular portion corresponds to a lower extremity of said human.

In an embodiment of the method, the particular portion corresponds to an upper extremity of said human.

In an embodiment of the method, the deciding involves how said particular portion broke said edge.

In an embodiment of the method, the deciding involves a speed with which said particular portion broke said edge.

In an embodiment of the method, the deciding involves an angle with which said particular portion broke said edge.

In an embodiment of the method, the deciding uses an interaction of said particular portion with another portion of said skeleton point cloud.

There is also described herein, in an embodiment, systems and methods for using a depth camera for detecting a patient falling in a manner partially obscured by a furniture object, the method comprising: obtaining a skeleton point cloud from an image of said depth camera, said skeleton point cloud being indicative of a human; defining an edge of an obscuring cloud within said image, said skeleton point cloud merging with said obscuring cloud due to a portion of said skeleton cloud interacting with said edge; determining said skeleton point cloud is still definable with said portion within a foreground of said obscuring cloud; and monitoring said skeleton point cloud for movement of said skeleton point cloud indicative of said human falling.

In an embodiment of the method, the edge comprises a generally vertical line.

In an embodiment of the method, the edge comprises a generally horizontal line.

In an embodiment of the method, the monitoring comprises gait analysis.

There is also described herein, in an embodiment, systems and methods for using a depth camera for detecting a patient falling in a manner partially obscured by a furniture object, the method comprising: obtaining a skeleton point cloud from an image of said depth camera, said skeleton point cloud being indicative of a human; defining an edge of an obscuring cloud within said image, said skeleton point cloud merging with said obscuring cloud due to a portion of said skeleton cloud interacting with said edge; determining said skeleton point cloud is obscured with said portion obscured by said obscuring cloud; and monitoring a non-obscured portion of said skeleton point cloud for movement of said non-obscured portion of said skeleton point cloud indicative of said human falling.

In an embodiment of the method, the edge comprises a generally vertical line.

In an embodiment of the method, the edge comprises a generally horizontal line.

In an embodiment of the method, the monitoring comprises reviewing for said horizontal line moving upward relative tot said skeleton point cloud.

### BRIEF DESCRIPTION OF THE DRAWINGS

FIG. 1 Provides a general block diagram showing an embodiment of the major components of a bed exit and fall inhibition and detection system.

FIG. 2 shows an example of a skeleton and associated cloud identified via a depth camera breaking line edges associated with a bed object.

FIG. 3 shows an example of a skeleton and associated cloud identified via a depth camera being broken by line edges associated with an object for a piece of furniture.

### DESCRIPTION OF THE PREFERRED EMBODIMENT(S)

The following detailed description and disclosure illustrates by way of example and not by way of limitation. This description will clearly enable one skilled in the art to make and use the disclosed systems and methods, and describes several embodiments, adaptations, variations, alternatives, and uses of the disclosed systems and methods. As various changes could be made in the above constructions without departing from the scope of the disclosures, it is intended that all matter contained in the description or shown in the accompanying drawings shall be interpreted as illustrative and not in a limiting sense.

Described herein are systems and methods which allow for machines to detect and predict bed exits and attempted bed exits, as well as a variety of other potentially problematic fall situations, using purely spatial recognition systems typically in the form of depth camera imagery. To put this another way, the systems and methods utilize depth cameras and other sources of “machine vision” as the primary or sole input to determine an attempted bed exit or a fall. This imagery can also use purposeful image obscuration techniques and low-light or non-visual light systems to obtain images which are not personally identifiable to the patient or meaningful to a human observer outside of the fall detection and inhibition arena. The systems and methods used herein will generally utilize machine observations and calculations during an attempted bed exit so as to eliminate much of the potential bias or relative skill that is currently used in conjunction with human observations and evaluations prior to a specific bed exit. However, while every patient is preferably assessed for risk at all times, in an embodiment the system the methods may only provide alerts for patients where a subjective heightened risk for falls has been previously identified to save resources.

In an embodiment, the systems and methods may issue warnings before the exit occurs, or detect the exit condition as it occurs, and alert against such conditions. These warnings may be used as notifications for healthcare personnel (for example, to initiate an additional action such as sending a nurse to the patient to assist), as notifications for the patient themselves (for example, triggering a warning to the patient to wait for assistance), or may trigger automatic mechanical responses to inhibit the attempt or assist in the exit (for example to raise a bed rail or to move a walker closer to the bed). All of these potential actions are generically referred to herein as “alerts”, “alarms”, or by similar terminology regardless of what actually occurs when an alert or alarm is triggered. The systems and methods may provide monitoring, assessment, and alerts to enable healthcare professionals to proactively intervene, and potentially prevent, adverse health events, including falls. These systems and methods, while particularly useful in acute healthcare facilities such as hospitals, are not limited to use there and may be used in other care facilities or even in a home setting to alert healthcare workers, aides, or family members in situations where fall risk is deemed to be a concern.

Throughout this disclosure, the term “computer” describes hardware which generally implements functionality provided by digital computing technology, particularly computing functionality associated with microprocessors. The term “computer” is not intended to be limited to any specific type of computing device, but it is intended to be inclusive of all computational devices including, but not limited to: processing devices, microprocessors, personal computers, desktop computers, laptop computers, workstations, terminals, servers, clients, portable computers, handheld computers, cell phones, mobile phones, smart phones, tablet computers, server farms, hardware appliances, minicomputers, mainframe computers, video game consoles, handheld video game products, and wearable computing devices including but not limited to eyewear, wristwear, pendants, fabrics, and clip-on devices.

As used herein, a “computer” is necessarily an abstraction of the functionality provided by a single computer device outfitted with the hardware and accessories typical of computers in a particular role. By way of example and not limitation, the term “computer” in reference to a laptop computer would be understood by one of ordinary skill in the art to include the functionality provided by pointer-based input devices, such as a mouse or track pad, whereas the term “computer” used in reference to an enterprise-class server would be understood by one of ordinary skill in the art to include the functionality provided by redundant systems, such as RAID drives and dual power supplies.

It is also well known to those of ordinary skill in the art that the functionality of a single computer may be distributed across a number of individual machines. This distribution may be functional, as where specific machines perform specific tasks; or, balanced, as where each machine is capable of performing most or all functions of any other machine and is assigned tasks based on its available resources at a point in time. Thus, the term “computer” as used herein, can refer to a single, standalone, self-contained device or to a plurality of machines working together or independently, including without limitation: a network server farm, “cloud” computing system, software-as-a-service, or other distributed or collaborative computer networks.

Those of ordinary skill in the art also appreciate that some devices which are not conventionally thought of as “computers” nevertheless exhibit the characteristics of a “computer” in certain contexts. Where such a device is performing the functions of a “computer” as described herein, the term “computer” includes such devices to that extent. Devices of this type include but are not limited to: network hardware, print servers, file servers, NAS and SAN, load balancers, and any other hardware capable of interacting with the systems and methods described herein in the matter of a conventional “computer.”

As will be appreciated by one skilled in the art, some aspects of the present disclosure may be embodied as a system, method or process, or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable media having computer readable program code embodied thereon.

Any combination of one or more computer readable media may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Throughout this disclosure, the term “software” refers to code objects, program logic, command structures, data structures and definitions, source code, executable and/or binary files, machine code, object code, compiled libraries, implementations, algorithms, libraries, or any instruction or set of instructions capable of being executed by a computer processor, or capable of being converted into a form capable of being executed by a computer processor, including without limitation virtual processors, or by the use of run-time environments, virtual machines, and/or interpreters. Those of ordinary skill in the art recognize that software can be wired or embedded into hardware, including without limitation onto a microchip, and still be considered “software” within the meaning of this disclosure. For purposes of this disclosure, software includes without limitation: instructions stored or storable in RAM, ROM, flash memory BIOS, CMOS, mother and daughter board circuitry, hardware controllers, USB controllers or hosts, peripheral devices and controllers, video cards, audio controllers, network cards, Bluetooth® and other wireless communication devices, virtual memory, storage devices and associated controllers, firmware, and device drivers. The systems and methods described here are contemplated to use computers and computer software typically stored in a computer- or machine-readable storage medium or memory.

Program code embodied on a computer readable medium may be transmitted using any appropriate medium, including but not limited to wireless, wireline, optical fiber cable, RF, etc., or any suitable combination of the foregoing.

Throughout this disclosure, the term “network” generally refers to a voice, data, or other telecommunications network over which computers communicate with each other. The term “server” generally refers to a computer providing a service over a network, and a “client” generally refers to a computer accessing or using a service provided by a server over a network. Those having ordinary skill in the art will appreciate that the terms “server” and “client” may refer to hardware, software, and/or a combination of hardware and software, depending on context. Those having ordinary skill in the art will further appreciate that the terms “server” and “client” may refer to endpoints of a network communication or network connection, including but not necessarily limited to a network socket connection. Those having ordinary skill in the art will further appreciate that a “server” may comprise a plurality of software and/or hardware servers delivering a service or set of services. Those having ordinary skill in the art will further appreciate that the term “host” may, in noun form, refer to an endpoint of a network communication or network (e.g., “a remote host”), or may, in verb form, refer to a server providing a service over a network (“hosts a web site”), or an access point for a service over a network.

Throughout this disclosure, the term “real time” refers to software operating within operational deadlines for a given event to commence or complete, or for a given module, software, or system to respond, and generally invokes that the response or performance time is, in ordinary user perception and considered the technological context, effectively generally cotemporaneous with a reference event. Those of ordinary skill in the art understand that “real time” does not literally mean the system processes input and/or responds instantaneously, but rather that the system processes and/or responds rapidly enough that the processing or response time is within the general human perception of the passage of real time in the operational context of the program. Those of ordinary skill in the art understand that, where the operational context is a graphical user interface, “real time” normally implies a response time of no more than one second of actual time, with milliseconds or microseconds being preferable. However, those of ordinary skill in the art also understand that, under other operational contexts, a system operating in “real time” may exhibit delays longer than one second, particularly where network operations are involved.

The present system is generally designed to provide information to a user that will allow a user to act on it in a certain proscribed manner. Specifically, to reduce a risk of injury from a fall by creating an accurate fall risk assessment at the time of an attempted bed exit for the risk associated with that bed exit. From the assessment, one can then ascertain whether certain movements constitute an attempted bed exit, the risk of that bed exit and, if an intervention before the exit occurs is deemed necessary, to reasonably inhibit the bed exit. At the same time, the systems can also limit the need to overly restrict behavior to inhibit falls by those who are still actively being monitored if the particular bed exit, or attempted bed exit, is deemed of sufficiently low risk. Thus, those with sufficiently low falling risk can be allowed freedom of movement while still being actively monitored for fall risk.

As the system and methods allow for the determination of instantaneous fall risk and can incorporate that information into an analysis of attempted bed exits, attempted bed exits for at-risk individuals can be responded to quickly without overly taxing resources and can permit intervention to prevent falls before they occur, while still allowing for generally increased freedom of motion (and improved quality of life) for most of the patient population.

As discussed herein, the system and methods utilize certain actions to determine and evaluate attempted bed exits by a patient in a hospital setting. That is, the systems and methods operate within a controlled environment and, as such, relate to predicting the likelihood of a fall while the patient is within that environment. While this is not required and any setting can utilize the systems and methods, these settings generally provide concerns for increased fall risk where attempted bed exits are most critical.

Throughout this disclosure, it should be recognized that there are generally two different types of issues related to falls. A person's fall risk is the likelihood that a person will fall at some time during their stay in an institution. Generally, any person that can stand is at a non-zero fall risk as even completely able-bodied individuals can trip and fall unexpectedly. This application is not primarily concerned with determining fall risk and preventing falls over the course of a stay. However, those with such an increased fall risk may be more likely to trigger an alert from the system and to trigger such an alert even in a situation where a patient with a reduced overall fall risk would not, due to that increased assessment of overall risk. The present systems and methods are primarily concerned with detection that an individual is in a situation where a fall is likely to occur in the very near future or has just fallen. In both these cases, aid can be provided to the individual quickly to both reduce or eliminate the fall actually occurring or to minimize it's health impact if it cannot be stopped.

To provide aid as quickly as possible after an alert is issued, it is generally important that the caregiver be notified by the alert in real-time or near real-time after the risk is detected. Further, because of the nature of the notification, a caregiver will generally need to act quickly on the notification, moving to the area where the patient is to assist them. Because of this, it is extremely important that a system for detecting falls not issue a large number of false positive detections. False positives can have the effect of “crying wolf” on the caregivers, and result in them not responding as quickly to an indication of a patient being likely to fall, resulting in a more negative outcome.

At the same time, a system for detecting falls is not particularly valuable if it generates false negatives. Further, where a false negative on fall risk is generated (or an alert does not result in sufficient response at sufficient speed) and the patient has already fallen, it is very important that the system detect this status quickly and also relay information that the fall has occurred to caregivers. If a patient falls and the fall is not detected close to the time the patient falls, the patient may not be able to move in a fashion that the system would detect as a patient, and, therefore, the system may not detect that the patient needs care for a very long time which could result in a very dangerous situation. In effect, a patient that has already fallen is essentially a very low fall risk because they are generally not standing and, therefore, cannot fall. Thus, if the initial fall is not detected as a fall, the system is unlikely to know to send caregivers at a later time that they are at increased risk. Detection of patient's who have already fallen is not discussed in depth herein, however, as this disclosure is primarily concerned with detection of patient's who are likely to fall in the near future, or who are falling.

One of the major concerns with fall detection systems is how machine vision can detect falls versus making sure those systems do not detect other motion in their field of view, for example a blanket falling off a bed, as a fall. It is often difficult for machine vision systems to identify and separate a human from other objects even though this is something that human vision is extraordinarily good at. To deal with difficulty in identifying humans, most machine vision systems accomplish the task of detecting falls by taking two actions. The first is to look for specific patterns which indicate normal human ambulation, shape, or movement within their field of view to identify a portion of the image as a human (and thus to be analyzed for falling). The second is to focus on those identified “human” portions of the image to detect the specific movement elements that are identified as indicative of falling within that portion.

This type of dual identification can be a very powerful tool, but it requires that the processors behind the machine vision systems be able to segregate what is human and what is not. In certain systems, for example those discussed in U.S. Pat. No. 10,453,202, the identification of human components of an image is aided by thermal imaging which may be used here. However, this disclosure is focused on identifying “skeletons” which are created when imaged clouds are determined to have expected human shape. The combined skeleton and associated cloud can then be considered as a human. This segregation can require substantial processing power compared to prior systems which can be why many prior systems are incapable of accomplishing it.

Regardless of how the imaging is performed, there are a number of difficult situations for machine vision to reconcile. These can generally be divided into two categories which correspond to the two determinations that the machine vision system makes. The first, and generally most common, category is situations where a portion of an image is not determined to be human. As image portions which are not identified as human are typically ignored (to avoid false negatives), an ignored component, even if it displays very distinct falling motion, will not trigger an indication of a human fall.

There are many ways that a human will not be identified as a human by a machine vision system but they all typically are because a human enters the machine's field of view not behaving as a human is expected to. For example, a human which first enters the machine's field of view while crawling would likely be identified as a non-human animal (e.g. a big dog) as it's movement is more akin to a dog than a human. Thus, such a human's movement would often be ignored unless and until the human stood up and began normal ambulation. Further, this situation is also true where the machine's view of a human is blocked or merges with another object for a period of time as the machine will typically forget that a portion of the image was human.

The latter situation is particularly concerning as humans often interact with other objects. For example, if a human sits down on a chair and doesn't move, they effectively merge with the chair and the human/chair object is typically seen by machine vision no longer as human, but as something not of interest. Most systems deal with this simply because when the human stands up and begins to move, that movement can be detected and the point source can be identified as human again.

However, it is desirable in fall detection, or other machine vision systems where identification of human objects from other objects is desirable, to provide for systems which are capable of identifying a specific portion of an image as human, and particularly as a human which is not behaving as a normal human, at the time they are first identified, or at a time they take a movement action, such as a fall, the occurrence of which is desired to be identified. This is why the present systems and methods will typically always be looking to locate clouds which can have skeletons associated with them. In this way, a cloud which is potentially merged may still include an identified skeleton and be observed. Similarly, a partial skeleton which is detected may also be associated with being human for purposes of additional observation.

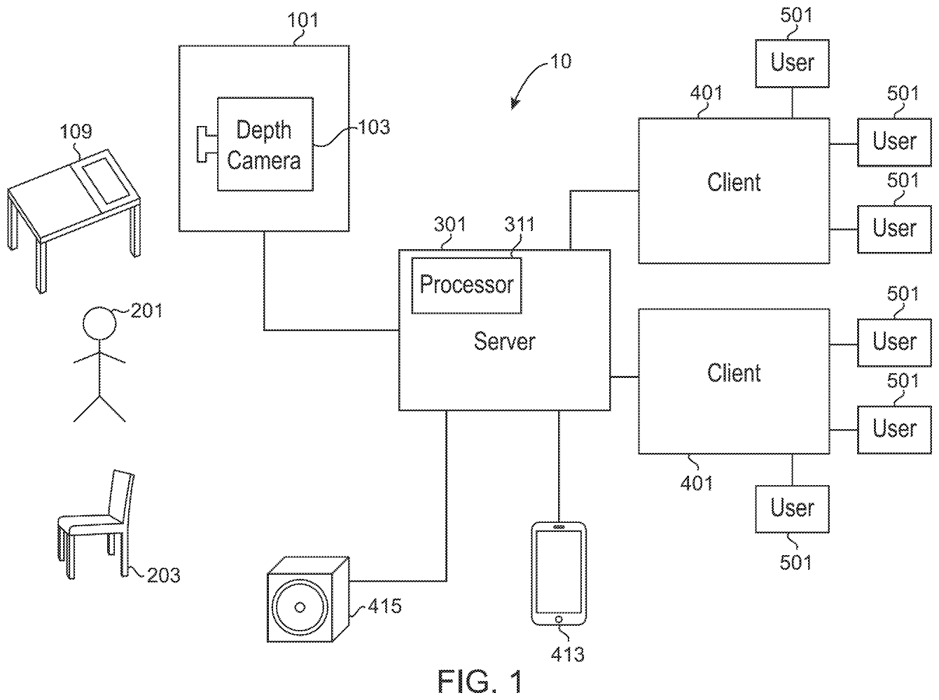

The fall detection methods discussed herein are generally performed by a computer system (10) such as that shown in the embodiment of FIG. 1. The system (10) comprises a computer network which includes a central server system (301) serving information to a number of clients (401) which can be accessed by users (501). The users (501) are generally humans who are capable of reacting to a fall as part of their job or task description. Thus, the users (501) will commonly be medical personal, corporate officers, or risk management personnel associated with the environment being monitored, or even the patient (201) themselves or family members or guardians. The users (501) could also be fully automated systems in their own right.

The system (10) can also provide feedback to mobile devices (413), such the smartphone of a patient's doctor who may not be currently at a computer. Similarly, information or requests for feedback may be provided to a patient (201) directly. For example, if a patient (201) is detected as having fallen, the system may activate a communication system (415) in the patient's (201) room asking them to indicate if they have fallen. This can allow a patient (201) to rapidly cancel a false alarm, or to confirm if they are in need of immediate aid.

Examining FIG. 1, in this embodiment, the primary, and often only, source of input about what is occurring in the room is a depth camera (101). The depth camera (101) which will typically monitor the patient directly but be disconnected from the patient (201) and from the bed (109). In an embodiment, the depth camera (101) may be a fixture in the room (for example being mounted to the ceiling or out of the way) so that it can monitor activity within the room. However, it may also be a temporary device which is brought into a specific room to handle fall detection for a specific patient. While a depth camera (101) will typically be used to monitor a patient, or at least a humanoid shape, it should be recognized that a depth camera (101) may also detect changes to the bed. For example the depth camera (101) may “see” compression of the bed from a user shifting their weight.

The depth camera (101), as the sole detector in the room, cannot rely on another sensor such as a bed sensor to determine the activity of a patient (201). Thus, the camera (101) as contemplated herein utilizes the interaction between specific forms of image processing to detect a bed exit. This form of image processing relates to the specific interactions of different kinds of imaged objects.

It should be recognized that this disclosure will discuss image analysis by a machine processor (311) using wording such as “recognizes” and other human concepts. It should be recognized by the reader that the machines herein do not need to process images in a manner similar to or even comparable to the manner they would be processed by a human observer visually watching the image. However, language which refers to such processing will typically utilize the processing by the human observer as a proxy representing the easiest way for a human reader to understand the processing that is occurring. For this reason, this disclosure should in no way be used to imply that the machines used herein are “sentient” even if they are ascribed human characteristics in the discussion of their decision making.

In an embodiment, the depth camera (101) will generally comprise an imager (103) or similar optics which takes video or similar image-over-time data to capture depth image data. Specifically, this provides for 3D “point clouds” which are representative of objects in the viewing range and angle of the camera (101). Operation of depth cameras (101) is generally well known to those of ordinary skill in the art and is also discussed in U.S. Pat. No. 9,408,561, the entire disclosure of which is herein incorporated by reference, amongst other places. In order to provide for increased privacy, the depth camera (101) may utilize silhouette processing as discussed in U.S. Pat. No. 8,890,937, the entire disclosure of which is herein incorporated by reference. To deal with monitoring at night or under certain other low light conditions, the depth camera (101) may utilize recording optics for recording the patient in an electromagnetic spectrum outside of human vision. That is, the camera (101), in an embodiment, may record in the infra-red or ultra-violet portions of the spectrum.

In an embodiment, the camera (101) utilizes an infrared (IR) sensitive camera (and particularly a near-infrared (NIR) camera) utilizing active imaging and an IR light source. This can allow for active imaging even at night by providing an NIR light source in the room and collecting images in primarily the NIR band. As the NIR light is not detected by the human eye, the room is still dark to the patient (201) while the NIR camera (101) can still image clearly.

While the depth capturing camera (101) can operate in a variety of ways, in an embodiment the camera (101) will capture an image and the processor (311) will obtain the images, in real-time or near real-time from the camera (101) and begin to process the images. Initially, foreground objects, represented as a three dimensional (3D) point cloud (a set of points in three dimensional space), can be identified from the depth image data using a dynamic background subtraction technique followed by projection of the depth data to 3D. Generally, objects in the foreground which are moving are considered to be of interest as these can potentially represent a patient (201) in the room. In FIG. 1, the image includes three foreground objects: the patient (201), bed (109), and a chair (203).

The depth camera (101) observing a hospital room or other acute care facility room will typically be able to resolve the image into points which form point clouds. These points are at a location in the x/y coordinates of the image, and are also at a depth (z coordinate) into the image. This structure can be used to find edges which allows for the detection of “clouds” or other separable “objects”. In effect, an object is commonly detected in machine vision due to its having an edge. That is, if one thinks about the depth that the point (representing the surface of something closest to an observer is from them) an object will have a sudden drop off at the edge of the object. As a simple example, The negative shape of a box can be seen by picking a fixed point and placing lines from a point of observation toward it as the edges of the box result in shorter lines than those that miss the box.

This quality of edge detection allows for the depth camera (101) to detect “clouds” or collections of points in the image. These clouds are effectively shapes where a majority of the points are much closer to the camera (101) than others which are off the cloud (over the edge). If one thinks of the longest distances as being the structure of the room (e.g. walls, ceiling, and floor), objects in the room will show up as clouds in the foreground.

These clouds can then be interpreted into objects of different types. For the purposes of this disclosure, the interpretation of clouds into objects is primarily focused on two different types of clouds. The first of these clouds is those that tend to imply man-made structures which are detected by linear or line edges although can also be detected by certain curves in alternative embodiments. One problem with detecting falls in hospital rooms, or in other rooms with beds (109), chairs (203), or similar pieces of furniture, is that there are generally a number of objects which can obscure the camera's (103) view of the patient partially or totally. For example, a bed (109) can obscure the lower extremities of a person that is walking behind it relative to the camera (101) positon. Further, an individual (201) walking in front of or lying on a bed (109) can actually have their point cloud merge with the cloud from the bed (109) while they are in front of it making a “hybrid” object with a single point cloud.

The furniture itself may also move under certain circumstances. For example, chairs (203) may roll on castors or curtains in the room may move in a breeze. Algorithms for detecting a patient (201) exiting a bed (109) or chair (203) can incorrectly detect a fall if the patient (201) has been in the bed or chair (203) for a period of time so that in the depth camera (101) image they appear merged with surrounding objects and have now become part of the background information unless the hybrid object is detected as a patient (201) on a bed (109). Alternatively, the chair (203) and patient (201) could have merged into a single foreground object due to their proximity even if the patient has continued to be in motion. This can make it difficult to detect which point cloud portion is the patient (201) versus the chair (203) as objects begin to separate when the patient (201) moves.

Objects merging into the background, or each other, in a depth camera (101) image is particularly problematic in fall detection should a fall occur while the point cloud image of the person is merged into the background or another object, partially obscured by another foreground object, or completely obscured by another foreground object. As the camera (101) evaluates moving objects, an object which is partially or totally merged or obscured at the time of falling may not be detected as a falling patient (201) as information of the movement is simply lost. For example, should a machine vision system be looking for movement of a patient's legs, if the legs are not visible at the time of fall (or have merged with another point cloud) the machine cannot detect the motion it is looking for to indicate a fall.

A major concern with failing to detect a fall, is if the machine vision system is looking to detect a fall as it happens, it is looking for movement which is not the same as a patient would have that has already fallen. Thus, if a patient falls and the fall is not detected, traditional systems do not look for a patient that has already fallen.

This type of false negative is particularly likely because the algorithms for fall detection generally work backward from the final fall position, to evaluate movement leading up to that position, to determine if a fall has occurred. That is, the algorithms recognize that a moving object (point cloud) is no longer moving, and thus go backward to evaluate the nature of the motion for a few seconds prior to the motion ceasing. If this movement is indicative of falling (e.g. it is downward) this can trigger a fall determination.

Further, it can be difficult for the camera (101) to determine that a partially blocked patient (201) has fallen because there is not necessarily enough walking movement or other information immediately prior to the fall for the camera (101) to use walking behavior or other algorithms to evaluate the general form and shape of the object to determine if it is a likely humanoid. The object may have been a foreground object of interest, but because of where it fell, it is not detected as a human falling as opposed to another object. Further, when methods such as those contemplated in U.S. patent application Ser. No. 12/791,496, the entire disclosure of which is herein incorporated by reference, for blurring of images and image edges to maintain privacy are being used, depth images can have a hard time determining a partial human shape versus the shape of an inanimate object such as a pillow, or a combination of both.

The vast majority of these false positives in fall detection are believed to result from two specific facets of the fall detection. The first is that the depth camera (101) image processing is generally looking at objects which are moving, and which then stop moving (at least in some dimensions or respects) in a position indicative of having fallen to the floor to locate a fall. The system then goes backward from the fall position, to evaluate the movement to see if it is indicative of a fall. Thus, falls which are partially blocked are problematic as the final position may not resemble an object on the floor which is not moving. Instead, the object disappears or merges at a higher position, and often will never reach the floor.

The second reason for false positives is that a patient (201) in the room which is not moving (such as when they are asleep) generally needs to become part of the background. If the camera (101) and processor (311) evaluated every object in the frame which was once moving, it would require a lot more computational power, but could also generate a large number of false signals. An object, such as chair (103), moved into the room should not be monitored simply because it once moved and the computation will become bogged down in useless information.

However, by allowing objects to become part of the background when they have not moved in a certain amount of time, it now becomes possible for the patient (201) to be lost in the image. This is most commonly because the patient (201) is now in a position (such as in bed (109) or sitting in a chair (203)) where they are not moving and their point cloud has merged with the cloud of the object (203). However, it can also occur when they are obscured by an intervening object as it means that their detection as an object of interest prior to disappearing from view, does not result in them automatically being an object of interest when they return.

The merging of point clouds creates the problem with object merging in the image.

Effectively, the patient (201) and chair (203) are now a single object to the depth camera (101) (or no object if they have become part of the background) after a period of time. When the patient (201) stands, the single object becomes two (or one with a part moving) and it is necessary to quickly assess which is the patient (201) and which is the chair (203). As many falls occur at the time that a stationary object starts moving (e.g. as the patient (201) stands from sitting) if the moving object (or object portion) is not quickly assessed as the patient (201), it is possible that the system (10) will not detect a patient (201) who stands and quickly falls. This creates a likely (and highly dangerous) false negative situation.

As indicated above, however, the problem is that a patient (201) that starts to stand from sitting and quickly falls, depending on the precise angle of the chair (203), can look very similar to a blanket on the patient's (201) legs slipping off and falling when a patient (201) turns in their sleep or in another type of movement that is not indicative of a fall. It is, thus, desirable to be able to determine where the patient is within a merged point cloud where a portion of the cloud beings moving. Basically, separating the cloud of the patient from the could of the furniture.

The merging of objects into background is particularly problematic with regards to the bed (109) and a patient (201) on the bed (109). Patients, particularly in hospital settings, that are on the bed (109) will often remain there for a substantial period of time because it is the primary piece of furniture in their room. Thus, the bed (109) is not only used for sleeping (which occupies a good portion of the day for everyone and a greater portion for those in an acute care setting), but also is often the primary object for doing activities such as eating, reading, or watching TV.

In the present disclosure, the system utilizes the depth camera (101) image and qualities of the point clouds to extract skeletons from clouds or parts of clouds in order to first identify humans in the room and to segregate them from other objects. The system (10) then utilizes the existence of lines at the edges of cloud, which are typically not part of the extracted skeleton, in order to detect furniture and other man-made objects. The interaction between the two types of objects may then be used to detect and predict falls.

Extraction of skeletons from a depth camera (101) image is already a known process. Simply, the human body has a relatively distinct shape characterized in that most humans have two legs attached to a torso that also has an attached head and two arms. This general structure of the human body is well recognized and can be seen in everything from children's drawings to abstract art. Because the human shape is relatively distinct compared to other shapes (most hospital rooms, for example, do not have humanoid artworks) the determination of a cloud, or even a portion of a cloud, which generally matches a humanoid shape in some position is a good way to detect a human.

It should be recognized that while the human body has a fairly distinct shape, some humans will have modifications to this shape, for example having less than two legs or two arms, but those are relatively uncommon, even in most hospital settings. Further, extraction of a skeleton typically does not require specific identification of all the elements of the body. Identification of some of them is often enough to determine that the shape is of a skeleton, in a particular position, and from that the skeleton can be drawn and associated with a point cloud or a portion of a point cloud at a different depth to other portions, but which conforms to the general shape of a skeleton.

Recognition that a point cloud generally has a skeleton shape allows it to be classified as a potential human in the room. Once so classified, the skeleton may be considered of greater interest than an object in the background and may not be allowed to fade into the background. Alternatively, the system could simply continuously check for skeletons in the image and simply assign that each is a human when detected, and then again when redetected, depending on the processing method.

In order to detect furniture and other objects in the room, the system will typically look for objects in the foreground which include lines as opposed to smooth curves. Most furniture includes lines representing edges as much constructed objects, and particularly those in more utilitarian settings such as acute care facilities, utilize lines in their construction. Lines are generally considered unnatural as few objects in nature appear linearly in a way that manufactured objects do. Thus, the presence of a relatively long line in an image is often indicative of a piece of furniture in a room.

The bed is a specific piece of furniture in an acute care room which is expected and is particularly linear in many aspects of its construction. Even in the most sparsely decorated hospital room, there is almost always a bed. Further, the bed can provide for a specific type of structure that can be detected by the depth camera which is the surface of the bed. This is near universally in the form of a rectangle and even from a variety of angles, and even if bent such as through the use of an adjustable bed, this rectangle can be identified, and maintained as the mattress surface and as the bed (109). U.S. Provisional Patent Application Ser. No. 63/530,209, the entire disclosure of which is herein incorporated by reference, provides for an exemplary method of bed surface detection.

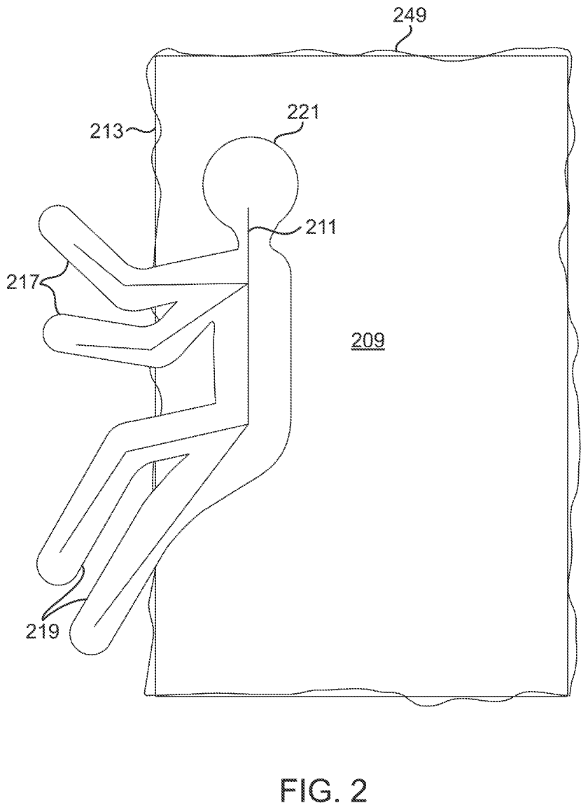

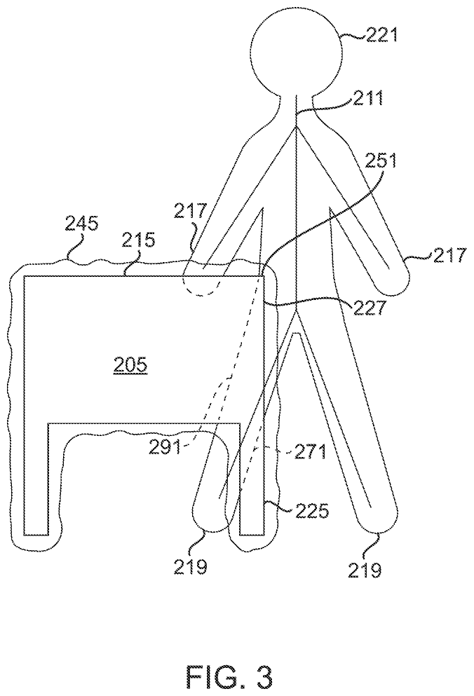

Once objects have been detected, they will typically be classified as either human skeletons, or as furniture, recognizing that human skeletons are of interest and their movement on position will often be analyzed, while furniture and other objects will be considered effectively background, but are also valuable for their interaction with humans. FIGS. 2 and 3 provide for two simplified images of how interactions between a human skeleton, and a furniture object can look.

As indicated above, the interaction between skeletons and the furniture objects provides for the ability to detect falls. In particular, a human will typically be at an increased risk of falling when standing from a sitting position and sitting will generally (in an acute care room) be on a chair (203) or the bed (109). A patient (201) sitting on the floor, for example, may be of interest as having already fallen, but is typically of little risk to fall. Further, a patient (201) who is on a bed (109) generally cannot fall if they don't stand up. While the patient (201) rolling off of a bed (109) is a possibility, this is typically unlikely in all but the most at risk patients (201). Instead, the act of standing implies a desire to ambulate, and falling requires the standing and/or ambulation to occur in order to happen. Thus, if a patient (201) can be detected prior to the act of standing (and, thus, prior to ambulating) the patient (201) can be provided with necessary assistance during the standing and ambulating to effectively prevent them from falling.

Examining FIG. 2, standing from a bed (109) typically first requires sitting on the bed (109). From sitting, standing is then typically accomplished. While a mostly prone patient (201) can move from a generally prone position to a standing position, this is unlikely. The act of sitting or getting ready to stand will typically involve an important action which is the moving of a portion of the body (namely the lower legs and feet) off the edge of the bed (109). The system (10), thus, looks for interaction of the point cloud (221) which has been identified as a skeleton (211) with a line (213) indicative of the edge of the bed (109) which is represented by a rectangle (209) from its own cloud (249). Specifically, the skeleton (211) breaking the line (213) representing the edge of the bed (109) at points such as (223) and (233) are looked for.

Breaking of the line (213) is effectively a first trigger action. Once this action has occurred, the system (10) will then typically further review the action of both what is happening with the skeleton (211) and its cloud (221) and the other object (209) and its cloud (249), and what has previously happened between the two clouds (221) and (249) or representations (211) and (209).

In the first instance, the system (10) will typically look to see from which direction the line (213) was broken. Specifically, as the bed as rectangle object (209) is in the foreground of the structure of the room (even if it is part of the background) it can generally be determined if the break occurred from the object (209), or onto the object (209). For example, should a nurse enter the room, the nurse will not be on the bed (109) when first detected as a cloud (221) and human skeleton (211) by the system. Further, the nurse will typically be easily detected as a skeleton (211) because of walking into the room, a very human activity. Should the nurse come over to the bed (109) and reach to a patient (201) on the bed (109) (who may also have been identified as another skeleton (211)) the nurse's skeleton will break the edge of the bed object (209) from external the bed object (209). This is not the situation shown in FIG. 2.

An external break will typically not be indicative of a potential fall concern as an external break does not imply an attempt to stand as the movement is backward from what would be expected in an attempt to stand. Instead, it is indicative of a person going to a safer position (prone as opposed to standing) or, as is the case here, the movement of a person that is not the patient (201). A further indication that this break is not movement indicative of falling is that the system may detect two skeletons (211) in the room. Most falls will occur when the patient (201) is alone in the room because if there is another person, that person will often readily help the patient (as they are likely a caregiver or family member). Thus, in this circumstance the break will generally not be considered a concerning event and no alert will be triggered.

It should be recognized that when the nurse backs up and moves to leave the room, the nurse will look like they are standing up from the bed (109). This is because, in some respects, they are carrying out a similar motion. However, as contemplated above, the skeleton of the nurse may have already been determined to not present a fall risk because they broke the edge (213) from external to the bed object (209). Further, as the nurse will generally have no reason to get completely on the bed (109), the edge (213) that they broke will typically not be restored until after they back away. Therefore, their backing away from the bed (109) does not result in a new edge break and would not generally be detected as an event of interest.

The above can be used to deal with breaks of the edge by a skeleton from external to the bed object (209) recognizing that those will generally not be activities of interest. Instead, for them to become an activity of interest, the skeleton will usually need to get completely on the bed (109) resulting in the line (213) representing the edge of the bed object (209) being restored and, therefore, any new break is from internal to the bed object (209).

A patient (211) getting up will typically break the edge (213) of the bed object (209) from internal to the bed object (209) in the process of getting up. It should be recognized that typically any break of the edge (213) by a skeleton (211) or cloud (221) internal to the bed object (209) may be treated as an activity of interest because any breaking of the line edge (213) of the bed object (209) from internal to the bed object (209) could imply a patient (201) falling from the bed (109), as the patient (201) could fall at any angle and in any way. However, the act of standing from an initially prone position on the bed (109) typically requires the patient (201) to move through a variety of positions in a particular order. Specifically the prone patient (201) will typically first move to a sitting position on the edge of the bed (109), and then move to a standing position from the sitting position. These transitions, even if performed fluidly and relatively quickly, will typically still take a measurable amount of time and it will typically be possible for a machine system to make the determination that a patient (201) is getting up in real time before they can actually complete getting up and while the process can still be interrupted.

A patient (201) getting up will typically first need to break the edge of the bed with their feet and lower legs (219). While this need not be the first or initial break, for example, their hands (217) may create a first break as they may put a hand on the edge of the bed (109) before their feet (219). Moving of the feet (219) off the edge of the bed (109) will still generally be necessary to go from a prone to a sitting or standing position. The system (10) will typically look to detect the motion of the skeleton (211) of the feet and/or lower legs (219) breaking the edge (213) as shown in, for example, FIG. 2. At this stage, the act of sitting or attempting to stand may be detected. However, it should be recognized that a sleeping patient (201) could move to a point where their feet and or legs are extended beyond the end of the bed and therefore this movement, while indicative, is not necessarily definitive of standing.

Instead, the system (10) will typically treat this detection as a trigger event which requires further processing. In this case, the processor (311) will typically look for prior movement corresponding to sitting or standing behavior, particularly motion which is known to be problematic. For example, should the hand (217) of the skeleton (211) interact with the foot (219) immediately prior to the break, that may be particularly concerning as it could imply the patient (201) is having to pull their leg over the edge which would be a strong indicator that their leg is insufficiently strong or controlled to support their weight should they attempt to stand. Further, when sitting, a patient (201) will typically not be extended but will end up with their upper legs arranged more under and in front of their torso such as is shown in FIG. 2. A sleeping patient (201) that happens to cause a break due to unintentional movement, however, will often be stretched out when they were to extend a leg (219) over the edge (213). Thus, the speed or angle that the leg (219) breaks the edge (213) may be used in an embodiment.

The above examples of detection of the edge break being interpreted as a sitting or standing event may be specifically known in an embodiment of the system (10), or they may actually be learned by the system (10). In an embodiment, the system (10) utilizes a neural network or other similar “artificial intelligence” (AI) programming to detect a sitting or standing event by simply training the system based on a large number of inputs with known outcomes. Which particular inputs used to determine that the motion is sitting or standing, or that the particular nature of the siting or standing motion is more or less likely to result in a fall, which may be deemed important by such a system (10), may be unknown and unknowable, but the system (10) will utilize some form of variables to determine if the movement of the patient (211) around the time of the break event is indicative, or not, of an attempt to sit or stand and generally if there are any indicators that this particular attempt is more or less likely than others to result in a fall.

Should the system (10) detect that a patient (201) is indeed attempting to sit, this becomes a point where an alert may be issued. In one embodiment, a nurse of other caregiver is warned of the action so that they can quickly review the same images and make a determination if an intervention is necessary. In an alternative embodiment, interventions may be automatically triggered based on the determination and/or alert. For example, the nurse may contact the patient (201) and ask them if they wish to get up and indicate that they should wait for assistance which is forthcoming. Alternatively, the same instruction may be given from a recording or other mechanical audio production.

It should be recognized from the above that the fall detection system (10) here has not detected an actual fall, and has not necessarily even detected an attempt to stand. Instead, the system has detected a movement by the patient (201) which is indicative of going to a sitting position at the edge of the bed (109) and of a potential future attempt to stand. However, this point of detection allows for intervention before standing and, generally, before falling, which allows for fall prevention as opposed simply to rapid fall detection and response.

While the above contemplates a situation where the potential for falls is detected early, it should be recognized that the interaction of a skeleton (211) and associated cloud (221) with line edges (213) can provide other features. In the discussion above, one thing that isn't mentioned is that the skeleton (211) and cloud (221) will typically be in the foreground compared to the bed object (209). This is because there is generally no reason for an individual to go under a hospital bed and with many types of hospital beds it is not actually possible to do so. A depth camera (101) will typically be able to recognize that the skeleton (211) is in front of the bed object (209) as the bed (109) will typically be represented by it's upper surface (the flat upon which the patient will lay).

Other furniture, however, and other items in the room may generate line edges and it is possible for a patient cloud (221) to be in the foreground compared to the object or have the object in the foreground compared to the patient cloud (221). In both situations, a similar detection to that contemplated above where the breaking of the line (213) by the skeleton (211) and/or cloud (221) can be detected can be used as a trigger for what may be an obscured fall.

One can consider that obscured falling can present two different situations which need to be detected. A patient (201) may fall in a manner that is partially obscured by another object in the room such as chair (203) according to two sub-situations. The first is that the user is obscured because another object (205) is physically in-between the camera (101) and the patient cloud (221). For example, this can occur if the patient (201) falls while standing behind a large chair (203) or their bed (109) for example. The second situation is where the patient cloud (221) is not directly obscured by the object (205) because they are actually between the object (205) and the camera (101), but the point cloud of the patient (221) has merged with the point cloud of the object (205) behind them prior to the fall occurring. This can occur, for example, when a patient (201) is sitting in a chair (203) facing the camera (101), and then falls straight forward. In this case, the patient's point cloud (221) will still be partially interconnected with that of the chair (203).

Both sub-situations can actually be handled the same, but the first will be discussed in conjunction with FIG. 3. In the first situation, the patient (109) falls in a manner that is obscured so that the depth camera (101) may not detect the movement as a fall. This type of concern can be addressed, in an embodiment of the invention, by teaching the system that disappearance of a skeleton (211) or of a large part of a skeleton (211) from the image quickly can be indicative of a fall. The system (10) can then reverse out of such disappearance if the manner of the disappearance is indicative of a fall. There are multiple ways of doing this. In a first, the partially obscuring object (205) corresponding to cloud (245) is considered to be primarily a horizontal line edge (215). That is, that it cuts off a horizontal “band” of the cloud (221). This, for example, would be the case for a bed (109) which is typically shorter than a patient (201) and, depending on position in the room, will often obscure or partially obscure the patient's (201) lower extremities when they are behind it (when it is between the patient (201) and the camera (101)). In most cases, a horizontally obscuring object is primarily of concern if it obscures the floor from the camera (101). This is because the camera (101) which can typically detect that a user is on the floor will evaluate if the motion to get to that position is indicative of a fall. However, if the floor is obscured, the trigger to evaluate the movement of the patient's point cloud (221) may not be activated because the triggering endpoint is not detected.

To detect that a patient (201) has fallen behind a horizontally obscuring object, the system will generally recognize that a sudden disappearance of a patient object (221) downward is cause for further evaluation. In effect, should the patient object (221) disappear in a manner which is sufficiently quick to be of interest, the system (10) will back up and evaluate the appearance of the disappearance. Falling behind an object (205), will typically result in a “line of obscuring” appearing to move upward over the patient object as the disappearance occurs resulting in an increase in obscured cloud (291). In effect, the system (10) can look for movement of the skeleton (211) or cloud (221) having a line or constant pattern image moving upward over it. While the motion does not need to be perfectly linear, as the human is falling downward and the object will often be non-moving, there will typically be an apparent upward movement of the top of the object (205) relative the patient cloud (221).

In such a scenario, the system (10) will typically trigger that a fall has occurred when such a pattern is seen. Further, the act of the horizontal line (215) “penetrating” the skeleton (211) or cloud (221) can be used as a trigger event. That is, when walking behind a chair back or bed, for example, the line of the object (205) will appear to go into the skeleton (211) or cloud (221) such as at point (251) which is generally toward the center of the cloud (221). It should be noted that the penetration is not direct as the depth of the two items is often different, but the term is still apt for discussion purposes. This “penetration” can used as the indicator that the cloud (221) and/or skeleton (211) is still there (e.g. at element (271)), but partially obscured. It should be recognized, in some respects, that this is the inverse of the bed edge (213) break. In that situation, the skeleton (211) or cloud (221) broke the edge (213), while in this embodiment it can be considered that the line edge (215) (or (227) as contemplated below) or associated cloud (245) broke the skeleton (211) and/or cloud (221).

One concern is that the falling patient (201) could pull the object down over themselves. In this situation, the upper surface of that object may not appear to move upward relative to the patient cloud (221) as the object will often fall at a similar rate to the patient (201) resulting in relatively little relative movement between the two. However, a falling patient (201) who grabs a stationary object and pulls it over on themselves, will typically only be able to pull it over after they have fallen some distance already. Thus, in reviewing the motion prior the disappearance of the patient point cloud (221), there should be a portion at the beginning where the upward movement of the upper surface of the object (205) relative to the patient (221) can be detected before the force of the fall results in the object (205) getting pulled over.

It should be recognized that while the above contemplates the horizontal line (215) present from the bed (109) or the top of a chair (203), the room may also include vertical edges (225). Chairs will typically have these also as indicated in FIG. 3, but they can also be present for doorways. With a vertical line edge (225), a process similar to the bed exit can be used. However, in this scenario, the system (10) will usually be interested in interactions that are both from external and internal to the object (205) and, again, they are interested in both foreground and background positioning. One element here is that the bed object (209) will typically not include any vertical lines. While the bed frame will provide for a vertical line in the image, the bed (109) having be previously identified via the upper surface rectangle as the object (209) will typically allow for the lower portion to effectively be ignored.

In the case of a vertical line edge (225), how the line is broken by the skeleton (211) or cloud (221), for example if is broken by a foot or leg (219) compared to a hand (217), is not as important as it generally is in the bed situation of FIG. 2. Instead, in this case, the breaking will typically be used to indicate that the skeleton (211) and/or cloud (221) is now interacting with a piece of furniture and can serve as an indicator to keep track of the skeleton (211), as a skeleton (211), even if not all of it is visible.

Again, in an embodiment, a neural network or other AI-type learning system can be used to determine motion of a partially obscured skeleton (211) which is indicative of a fall which is partially obscured by an object (205). Further, should the skeleton (211) disappear completely behind the object, a simple calculation of how long it remains out of view can be used to determine if a potential warning situation exists. Should the skeleton (211) disappear for a period of time that is deemed too long, the system (10) can, for example, call out to the patient (109) to see if they are OK. If no response is received, that can comprise an alert situation. If a response is received, the nature of the response can be analyzed to verify that the patient (109) is indeed OK and is simply out of view. This can then be used to reset the time clock.

One concern in any visual monitoring system is that there is a possibility that there are actually multiple skeletons (211) detected in the room and determining which is the patient (201) could be an issue. In the first instance, with regards to fall detection, if multiple skeletons (211) are detected determining which is the patient (201) may be unnecessary. Should the skeletons (211) be in proximity, where it may be difficult to determine which is the patient (201) and which is a family member or staff member, it is highly unlikely that the patient (201) will fall precisely because that other individual is in close proximity and likely helping to steady them or watch them. Even if the skeletons (211) are separate, it is still likely that the other individual the room would be available for immediate help should it be required and therefore fall concerns are greatly reduced.

In the event that segregation of patient skeletons (211) from other skeletons (211) be required, one option is simply to store where the patient skeleton (211) was last observed or to treat a skeleton (211) that is behaving more like a patient (e.g. that was first detected getting off the bed as opposed to from a chair) as more likely the patient (201). In an alternative embodiment, the possibility of fall for any skeleton (211) can simply be watched for and should a potential fall be detected an alert be sent. Should the fall actually be a non-patient, this is not necessarily a problematic false positive as such detection may provide confidence by others in the effectiveness of the system (10) and detect an alternative dangerous situation. It may also handle a situation where a room is shared by multiple patients.

As contemplated above, depending on the particular embodiment, how the system (10) may determine the fall risk may utilize neural networks, statistical calculations, spreadsheets, or any other forms of variables that the system (10) has determined to be informative for the monitored facility and patients (201). However, these will generally fall into the broad category of being related to specifics of a patient's detected movement during the process of rising.

To further refine the detection, the system, in an embodiment, may combine the data from the depth camera (101) with other criteria either from entered records which are accessed when the patient is identified (e.g. that a doctor has indicated that the patient is currently connected to an IV or an initial check in screening indicated that the patient walks with a walker) or can be obtained from image data. This allows for specifics of known risk factors related to external influencers on the patient (e.g. them awakening from the effects of anesthesia versus arising after natural sleep) or them already being known to walk with difficulty to be combined with image data. For example, a patient (201) attempting to rise or exit may be detected and data (313) loaded from the patient (201) may indicate that they walk with a walker. The image data may be scrutinized for an object that appears to be of the correct shape to that of a walker being near the point where they are attempting to rise. Similarly, a bar of metal being detected as obscuring a patient's legs at the point one would expect the seat of a walker to be located would indicate that they have their legs down inside their walker.

In addition to the above, additional criteria to be examined can include if it is typical or expected for this individual to be currently attempting a bed exit along with the length of bed occupancy may also tied to the exit. A patient (201) who has spent a long period of time in bed will likely have an increased fall risk and a patient (201) who regularly gets up may also have an increased fall risk as they are simply more exposed to the possibility of falls. Similarly, a patient (201) who is getting up atypically for that particular patient (201) may also have an increased risk. For example, an individual getting up at night who has not gotten up at night before may be at an increased risk due to darkness. However, these criteria (or any subset of the criteria) can be evaluated without any prior data, processed through a machine learning algorithm as contemplated above and compared with data compiled over a number of years. The system server (301) can determine when the particular values are being obtained for an individual it has evaluated before (and therefore provide results based on that particular individual) but can also assess the data for macro patterns within groups or the population of individuals (e.g. for males over the age of 65 who have had hip replacements).

Once the instantaneous fall risk has been evaluated, the risk can be presented to a user (501) through a variety of interfaces (401). In a hospital setting, for example, the instantaneous fall risk of a patient (201) may be provided to a nurse's monitoring station for a floor or wing of a hospital in real-time or near real-time to allow them to quickly respond to a potentially high risk situation being detected. This allows the nurses to know that a patient (201) is active and, in the event they are determined to be a higher fall risk, may need nursing or other staff to go assist them in a very short time frame. Alternatively, the patient (201) themselves may receive a notification by the system (10) to inhibit risk. For example, an interface may provide a recorded statement to the user (501) via a speaker (415) telling them to stay seated on the bed (109) and not attempt to stand until a nurse arrives to avoid falling. When this is combined with quick nurse response (due to their notification by the alert) the patient (201) will typically be more likely to be willing to wait for assistance. The system (10) can also provide for mechanical intervention. This can include actively restraining a patient (201) at particularly high fall risk until a nurse can arrive or can be as simple as illuminating the room at night to decrease the risk due to darkness.

Taken together, it can be possible based upon data obtained in real time from just a depth camera (101), to determine whether a bed exit is being attempted, and whether such an exit is more likely (i.e., being conducted by an individual with a high risk of falling) or less likely to result in a fall. If the attempted exit is considered sufficiently risky (based on any desired criteria), an alert can be issued which can be evaluated by appropriate staff, and where necessary, an intervention can occur. If the attempted exit is not determined to be of sufficient risk, staff need not be tasked to intervene and the patient's freedom of movement need not be curtailed.

An attempted exit can result in a successful exit quickly for some patients. Often a quick ability to exit will actually indicate a decreased risk of fall. In that situation, the system (10) can determine whether, following the successful exit, a walking sequence is identifiable. Whether an exit is indicated, a walking sequence is identified, and a bed is not occupied (based upon path history of an object moving away from the bed at the time of the bed exit), it can be assumed it is a patient walking and not falling. This too can result in either action or inaction of staff and/or issuance of an alert at all. Such an evaluation is particularly useful for individuals who are at an intermediate fall risk or may be expected to be improving their risk over time. For example, a patient may be at high risk as they come off of anesthesia, but over time that risk will decrease, and the system may take their actual behavior into account in determining if their risk concern should be reevaluated or altered.

While the invention has been disclosed in conjunction with a description of certain embodiments, including those that are currently believed to be useful embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

It will further be understood that any of the ranges, values, properties, or characteristics given for any single component of the present disclosure can be used interchangeably with any ranges, values, properties, or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

The qualifier “generally,” and similar qualifiers as used in the present case, would be understood by one of ordinary skill in the art to accommodate recognizable attempts to conform a device to the qualified term, which may nevertheless fall short of doing so. This is because terms such as “spherical” are purely geometric constructs and no real-world component or relationship is truly “spherical” in the geometric sense. Variations from geometric and mathematical descriptions are unavoidable due to, among other things, manufacturing tolerances resulting in shape variations, defects and imperfections, non-uniform thermal expansion, and natural wear. Moreover, there exists for every object a level of magnification at which geometric and mathematical descriptors fail due to the nature of matter. One of ordinary skill would thus understand the term “generally” and relationships contemplated herein regardless of the inclusion of such qualifiers to include a range of variations from the literal geometric meaning of the term in view of these and other considerations.

1. A method for using a depth camera for detecting a patient attempting to leave a furniture object, the method comprising:
obtaining a merged point cloud from an image of said depth camera, said merged point cloud being indicative of a human on a furniture object;
reviewing said image to locate a within said merged point cloud a skeleton point cloud indicative of said human;
defining an edge of said merged point cloud which is not part of said skeleton point cloud, said edge being generally linear;
monitoring said merged point cloud for said skeleton point cloud to move and, by moving, break said edge; and
using said break to determine that said human is attempting to leave said furniture object.

2. The method of claim 1, wherein said furniture object comprises a chair.

3. The method of claim 1, wherein said furniture object comprises a bed.

4. The method of claim 1, wherein, during said using, said determination involves deciding if a particular portion of said skeleton point cloud moved and broke said edge.

5. The method of claim 4, wherein said particular portion corresponds to a lower extremity of said human.

6. The method of claim 4, wherein said particular portion corresponds to an upper extremity of said human.

7. The method of claim 4, wherein said deciding involves how said particular portion broke said edge.

8. The method of claim 7 wherein said deciding involves a speed with which said particular portion broke said edge.

9. The method of claim 7 wherein said deciding involves an angle with which said particular portion broke said edge.

10. The method of claim 4, wherein said deciding uses an interaction of said particular portion with another portion of said skeleton point cloud.

11. A method for using a depth camera for detecting a patient falling in a manner partially obscured by a furniture object, the method comprising:
obtaining a skeleton point cloud from an image of said depth camera, said skeleton point cloud being indicative of a human;
defining an edge of an obscuring cloud within said image, said skeleton point cloud merging with said obscuring cloud due to a portion of said skeleton cloud interacting with said edge;
determining said skeleton point cloud is still definable with said portion within a foreground of said obscuring cloud; and
monitoring said skeleton point cloud for movement of said skeleton point cloud indicative of said human falling.

12. The method of claim 11, wherein said edge comprises a generally vertical line.

13. The method of claim 11, wherein said edge comprises a generally horizontal line.

14. The method of claim 11, wherein said monitoring comprises gait analysis.

15. A method for using a depth camera for detecting a patient falling in a manner partially obscured by a furniture object, the method comprising:
obtaining a skeleton point cloud from an image of said depth camera, said skeleton point cloud being indicative of a human;
defining an edge of an obscuring cloud within said image, said skeleton point cloud merging with said obscuring cloud due to a portion of said skeleton cloud interacting with said edge;
determining said skeleton point cloud is obscured with said portion obscured by said obscuring cloud; and
monitoring a non-obscured portion of said skeleton point cloud for movement of said non-obscured portion of said skeleton point cloud indicative of said human falling.

16. The method of claim 15, wherein said edge comprises a generally vertical line.

17. The method of claim 15, wherein said edge comprises a generally horizontal line.

18. The method of claim 17, wherein said monitoring comprises reviewing for said horizontal line moving upward relative tot said skeleton point cloud.
